(19) United States
(12) Patent Application Publication
(10) Pub. No.: US 2021/0052506 A1
(43) Pub. Date: Feb. 25, 2021

(54) BIOXOMES PARTICLES, REDOXOMES, METHOD AND COMPOSITION

(71) Applicant: ORGENESIS INC., Germantown, MD (US)

(72) Inventor: Sabina GLOZMAN, Naharya (IL)

(73) Assignee: ORGENESIS INC., Germantown, MD (US)

(21) Appl. No.: 17/046,058

(22) PCT Filed: Apr. 4, 2019
(86) PCT No.: PCT/IL2019/050391

§ 371 Date: Oct. 8, 2020

Related U.S. Application Data
(60) Provisional application No. 62/654,771, filed on Apr. 9, 2018.
(60) Provisional application No. 62/794,859, filed on Jan. 21, 2019.

(51) Int. Cl. A61K 9/50; B01D 11/02

(52) U.S. Cl. CPC A61K 9/5068; A61K 45/06; B01D 11/0203

(57) ABSTRACT

Provided an artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source; methods of use of the particles; and processes for manufacturing thereof.

### FIELD OF THE INVENTION

The invention relates to novel bioxome particles engineered to deliver active biomaterial, methods for their preparation and uses thereof.

### BACKGROUND OF THE INVENTION

Exosomes are naturally-occurring, secreted, lipid membrane micro-vesicles that carry nucleic acids and proteins, enabling intercellular communication by transfer of these materials between cellular organelles and between the cells. Exosomes are formed by invagination of endolysosomal vesicles that are released extracellularly upon fusion with the plasma membrane.

Exosomes have various physiological functions under homeostatic conditions or during pathology of a disease state. Cells release into the extracellular environment diverse types of membrane vesicles of endosomal and plasma membrane origin called exosomes and micro-vesicles (MV), respectively. These, collectively, extracellular vesicles (EVs) represent an important mode of intercellular communication by serving as vehicles for transfer between cells of membrane and cytosolic proteins, lipids, and RNA.

Lipids are a particularly valuable substrate for FRs due to the multiple double bonds present in fatty acids, especially polyunsaturated fatty acids (PUFA). Lipid peroxidation (LPO) is a chain process, consisting of three main steps: initiation, propagation and termination. Main component of natural plasma membrane are polar phospholipids (PL) that consist of PUFA and are therefore vulnerable to oxidative stress. Traditionally, LPO has been regarded as the major process that produces damage from oxygen radicals leading to membrane destruction, degeneration processes, and cell death.

Presence of genetic material and protein in natural exosomes implies that exosome might serve as a vehicle for such biological materials. Structure of membrane bilayer and aqueous core, similar to that of the liposome, enables delivery of their contents across cellular membranes. Thus, exosomes have great potential to serve as delivery systems for various biomaterials. Numerous application of exosomes and methods for exosome preparation are described in US2004/0082511; U.S. Pat. Nos. 5,428,008, 5,165,938; US 2004/0082511; U.S. Pat. No. 9,119,974; US2013/0143314; US2011/0014251; US2013/0052647; WO2015110957; WO/2015/138878; US20130209528; WO2009105044; U.S. Pat. Nos. 8,138,147; 8,518,879; 8,138,147; US2011/0003008; US2013/0209528; US2011/0003008; US2013/0209528;

Based on its membrane fusion and intracellular targeting features, exosomes holds promise to apply as Drug Delivery System in order to overcome unsolved need in currently used in the state of the art DDS: (i) instability of naked gene and nucleic acids delivery due to extracellular enzymes; (ii) viral and liposome DDS are recognized by the host immune system as foreign particles resulting in generation of antibodies against them and thereby decreasing delivery and safety; (iii) majority of active natural and therapeutic drugs are hydrophobic in nature, therefore prone to LPO and have poor bio-availability. The all above are major challenges to overcome in order to fulfill exosome-DDS (drug delivery system) promise are to improve yield of production, control loading, stability and composition, including proteins and DNA. In addition, currently known methods for exosome production are complex and multistep, that limit their clinical applicability as delivery vehicles for therapeutic cargo. Thus, there is still a need in simple, robust, cost effective, industrial method for large scale production of exosome-inspired artificial membrane vesicles which will maintain maximal membrane integrity, stability towards LPO chain reaction, and natural characteristics required for their use as therapeutics; delivery vehicles and research tools.

### SUMMARY OF THE INVENTION

Accordingly, it is a principal object of the present invention to overcome disadvantages of the prior art methods and systems for industrial-scale production of exosomes-like artificial particles which maintain maximal membrane integrity and engineered for use as vehicles for delivery of active biomolecules or as standalone agents having multiple industrial applications.

The invention provides an artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source.

The invention further provides a composition comprising an artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source, and at least one carrier.

The invention further provides a process for the manufacture of a sample comprising a plurality of bioxome particles, wherein the bioxome particles are engineered to carry a cargo comprising at least one active molecule and designed to undergo fusion with a target cell to release the cargo; and wherein said bioxome particles comprise a cell membrane component derived from a selected cellular or extracellular source; the process comprising:
a. Performing total cell lipid extraction from the selected cellular or extracellular source in a mild solvent system to obtain a lipid extract;
b. Drying the lipid extract; and
c. Inducing self-assembly of bioxome particles by performing at least one step of ultra-sonication;
   wherein the resulting bioxome particles in the sample are characterized by an average particle size of around 0.03 μm to 5 μm.

The invention further provides a method of treating or preventing a pathology in a subject in need of such treatment, comprising administering to the subject the pharmaceutical composition comprising an artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source, and at least one carrier.

The invention further provides a method of improving a skin condition in a subject in need comprising administering to the subject the composition comprising an artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source, and at least one carrier.

### BRIEF DESCRIPTION OF THE DRAWINGS

FIG. 1. Particle size distribution of bioxome particles as measured by Malvern instruments: A. immediately after the isolation B. a month following isolation;

FIG. 2. Bioxome particle size distribution. A. particle size distribution of the bioxome measured by ZetaSizer nano analyzer. The measurements by the analyzer are represented with two peaks on the histogram (x axes represent particle size). B. particle size distribution of the bioxome detected by the NanoSight particle size analyzer. X-axes represent particle size in nm, y-axes represent the number of particles per ml. Left panel represent 3 measurements of the same sample. Right panel represents the average of the 3 measurements performed on the left;

FIG. 3. Bioxomes stability and particle size distribution. Presented are the size distribution profiles of bioxome produced from 3 different samples. A. Sample without re-ultrasonication. B. Sample with re-ultrasonication. C. Sample following lyophilizing and ultrasonication;

FIG. 4. Confocal microscope image of fusion of BioDipy labeled Bioxomes into Human Foreskin Fibroblasts primary culture (HFF). Bioxomes produced from three various cell sources and average diameter of particle sizes as measured 24 hours before the experiment. A. Primary Human Umbilical Vein Endothelial Cells (HUVEC)-ATCC® PCS-100-010™ Particle size: >90% ˜1.4 mcn; B. Primary Human Mammary Epithelial Cells; ATCC® PCS-600-010™. Particle size measured 24 hours before the experiment: 40%-˜300 nm; 60%: 600 Particle size; measured 24 hours before the experiment −1 mcn nm. C. NIH3T3 fibroblasts; ATCC® CRL-1658; Particle size: >90%˜750 nm;

FIG. 5. general scheme of isolation of bioxome particles from adherent cells;

FIG. 6. Specificity of bioxome particles to their origin-target tissue. A. schematic representation of the procedure. B. experimental data;

FIG. 7. Schematic representation of a Redoxome particle;

FIG. 8. Redox sensitivity of redoxome particles by measuring POBN adduct formation (EPR spectra) in two different concentrations of DHA, and in the presence of α-Tocopherol;

FIG. 9. A. Kinetics of hydroxyl radical-induced leakage from redoxome particles. B. Triton-induced calcein leakage from redoxome particles; and

FIG. 10. RNA encapsulation capacity. Bioxome encapsulated with co-extracted RNA produced from human mesenchymal stem cells (MSCs) derived from bone marrow (PromoCell, C-12974), the size measurements performed by the NanoSight analyzer. A. repetitive (three times) freeze-thaw stability as a loading enhancement approach, lost uniformity, but stay in spec <1.5 mcn. B. RNA uniform encapsulation post single freeze-thawing cycle and C-without freeze thawing cycle-measured fresh after ultrasonication/RNA encapsulation.

### DETAILED DESCRIPTION OF THE INVENTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The invention provides an artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source. As used herein, the term “bioxome” refers, without limitation to an artificial, submicron nano-particle having resemblance to natural extracellular vesicles (EV). The particle size of the bioxome of the invention ranges from 0.03 μm to 5 μm. In one embodiment, the size of the bioxome is 0.1-0.7 μm; 0.1-0.5 μm, 0.2-0.5 μm; 0.3-0.5 μm. In another embodiment, the average particle size is 5 μm or less; 1.5 μm or less; 0.7 μm or less; 0.5 μm or less; 0.3 μm or less; 0.15 μm or less. In one embodiment, the average particle size is 0.5 μm to 1.5 μm. In one embodiment, the average particle size is 0.4 μm to 0.8 μm. In another embodiment, the average particle size is 0.3 μm to 0.5 μm. In yet further embodiment, the average particle size is 0.4 μm to 1.5 μm when particle size is measured within few hours after the preparation. In yet another embodiment, the particle size is 0.8 μm to 5 μm when particle size is measured within a month after the preparation and bioxome particles are stored at 0° C. to −4° C.

In one embodiment, the bioxome particle is free of load. In yet another embodiment, the particle is carrying a cargo comprising at least one active molecule. In one embodiment the cargo comprises at least two active molecules. In another embodiment, the cargo comprises a plurality of active molecules. As used herein, the term “active molecules” refers, without limitation to signaling molecules, biomolecules; genetic and translation modifying nucleic material; deoxyribonucleic acids (DNA); ribonucleic acids (RNA); organic molecules; inorganic molecules; amino acids; vitamins; polyphenol, steroid, lipophilic poor soluble drug, vasomodulators; peptides; neurotransmitters and analogues of thereof; nucleosides; proteins, including without limitation growth factors, hormones, aptamers, antibodies, cytokines, enzymes, and heat shock proteins; or any other molecules that can exert biological function. In one embodiment, the active molecule is a cannabinoid,cannabinoid acid and endocannabionid. In yet another embodiment, the cannabinoid or cannabinoid acid is selected from the group consisting of tetrahydrocannabinolic acid (THCa), cannabidiolic acid (CBDa), cannabinolic acid (CBNa) cannabichromenic acid (CBCa), tetrahydrocannabinol (THC), cannabinol (CBN), cannabidiol (CBD), and cannabichromene (CBC), and endocannobinoids and analogues of thereof. According to one embodiment, the nucleic acid is ribonucleic acid (RNA) or deoxyribonucleic acid (DNA). According to further embodiment, the nucleic acid is RNA, and is selected from the group consisting of siRNA, an antisense RNA, iRNA, microRNA, an antagomir, an aptamer, and a ribozyme mRNA, or any combination thereof. According to one embodiment, the active molecule has a therapeutic effect. As used herein, the term “therapeutic effect” refers, without limitation to response(s) after a treatment of any kind, the results of which are judged to be useful or favorable. This is true whether the result was expected, unexpected, or even an unintended consequence. In one embodiment, the therapeutic effect is selected from the group consisting of anti-inflammatory effect, anti-fibrotic effect, anti-tumor effect, and neuroprotective effect. A non-limiting list of selected cargo for combating inflammation, fibrosis, hyperglycolisis, such as diabetic nephropathy, comprises naturally derived ubiquitous phenolic compounds, such as ferulic acid, natural phenols, for example resveratrol, rutin, quercetin, phenolic acids, vitamins, and allicin, cannabinoids selected from the group consisting of tetrahydrocannabinolic acid (THCa), cannabidiolic acid (CBDa), cannabinolic acid (CBNa) cannabichromenic acid (CBCa), tetrahydrocannabinol (THC), cannabinol (CBN), cannabidiol (CBD) and cannabichromene (CBC) or/and the derivatives of thereof, and synthetic analogues, dexmedetomidine, (α2-AR) agonist, metabolic protectants, including, without limitation, vildagliptin, pristimerin, metformin, pyridoxamine, vasomodulators, epinephrine, rutin, isoxsuprine. The non-limiting list of cargo exerting anti-tumor effect comprises, without limitation, chemotherapeutic agent, including without limitation cisplatin, carboplatin, chlorambucil, melphalan, nedaplatin, oxaliplatin, triplatin tetranitrate, satraplatin, imatinib, nilotinib, dasatinib, and radicicol; an immunomodulatory agent, an antiangiogenic agent, a mitotic inhibitor, a nucleoside analog, a DNA intercalating agent, anti-ageing agents, dipeptides, epigenetic factors and modulators, metformin, rapamycin, valproic acid or salt, wortmannin, polyamine spermidine, HDAC inhibitors sodium butyrate, butyric acid, sirtuin activators, resveratrol, co-enzyme CoQ1, small dicarboxylic acids, aspirin, salicylic, benzoic acid, carnitine analogues, human growth hormone, a topoisomerase analogue, an antibody, a cytokine, a folate antimetabolite, anti-glycolisis agent, inhibitor oligonucleotide to a human oncogenic or proto-oncogenic transcription factor; or chemotherapeutic agent, an immunomodulatory agent, an antiangiogenic agent, a mitotic inhibitor, a nucleoside analogue, a DNA intercalating agent, a topoisomerase analog, an antibody, a cytokine, or a folate antimetabolite, hexokinase inhibitor, a lactate dehydrogenase inhibitor, a phosphofructokinase 2 or phosphofructo-2-kinase/fructose-2,6-bisphosphatase inhibitor, a pyruvate kinase M2 inhibitor, a transketolase inhibitor, a pyruvate dehydrogenase inhibitor, a pyruvate dehydrogenase kinase inhibitor, a glucose-6-phosphate dehydrogenase inhibitor, a GLUT inhibitor, a proton transport inhibitor, a monocarboxylate transporter inhibitor, a hypoxia-inducible factor 1 alpha inhibitor, an AMP-activated protein kinase inhibitor, a glutamine inhibitor, an asparagine inhibitor, an arginine inhibitor, a fatty acid synthase inhibitor, an ATP-citrate lyase inhibitor, dimethyl malate, and malic enzyme 2 inhibitor, or any combination thereof.

In one embodiment of the invention, the cellular or extracellular source is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, postnatal umbilical cord, placenta, amniotic sac, kidney tissue, neurological tissue, adrenal gland tissue, mucosal epithelium, smooth muscle tissue, a bacterial cell, a bacterial culture, fungi, algae, a whole microorganism, conditional medium, amniotic fluid, lipoaspirate, liposuction byproducts, fecal sample, and a plant tissue.

In one embodiment, the bioxome is a redoxome. As used herein, the term “redoxome” refers, without limitation, to bioxome particle carrying a cargo comprising at least one redox active, free radical scavenging compound. In one embodiment, the release of a packaged complex from the redoxome particle blocks the LPO chain reaction. In yet further embodiment, the release of a packaged complex from the redoxome particle is preferentially stimulated at sites of oxidative stress. In one embodiment, the redoxome is capable of blocking LPO chain reaction, by means of, without limitation, lipid radical/peroxide trap, such as vitamin E, terpenoids, polyphenols, flavonoid, phenolic acids, cannabinoids, retinoids, vitamin D, lipoic acid, sterols. According to one embodiment, the redoxome comprises fenton reaction complex blockers, hydroxyl radical trap, iron chelator and a lipid radical trap. In one embodiment, the radical trap is ascorbic acid, nitric oxid donor (S-nitrosoglutathione), or a derivative thereof. A non-limiting list of iron chelators of the invention comprises, without limitation, desferrioxamine (DFX), ethylenediaminetetraacetic acid (EDTA), rutin, disodium EDTA, tetrasodium EDTA, calcium disodium EDTA, diethylenetriaminepentaacetic acid (DTPA) or a salt thereof, hydroxyethlethylenediaminetriacetic acid (HEDTA) or a salt thereof, nitrilotriacetic acid (NTA), acetyl trihexyl citrate, aminotrimethylene phosphonic acid, beta-alanine diacetic acid, bismuth citrate, citric acid, cyclohexanediamine tetraacetic acid, diammonium citrate, dibutyl oxalate, diethyl oxalate, diisobutyl oxalate, diisopropyl oxalate, dilithium oxalate, dimethyl oxalate, dipotassium EDTA, dipotassium oxalate, dipropyl oxalate, disodium EDTA-copper, disodium pyrophosphate, etidronic acid, HEDTA, methyl cyclodextrin, oxalic acid, pentapotassium, triphosphate, pentasodium aminotrimethylene phosphonate, pentasodium pentetate, pentasodium triphosphate, pentetic acid, phytic acid, potassium citrate, sodium citrate, sodium dihydroxyethylglycinate, sodium gluceptate, sodium gluconate, sodium hexametaphosphate, sodium metaphosphate, sodium metasilicate, sodium oxalate, sodium trimetaphosphate, tea-EDTA, tetrahydroxypropyl ethylenediamine, tetrapotassium etidronate, tetrapotassium pyrophosphate, tetrasodium etidronate, tetrasodium pyrophosphate, tripotassium EDTA, trisodium EDTA, trisodium hedta, trisodium NTA, trisodium phosphate, malic acid, fumaric acid, maltol, succimer, penicillamine, dimercaprol, deferipron, a natural protein based iron chelator, melatonin, siderphore, zinc or copper cation, or salt or complex, and desferrioxamine mesylate, or a combination thereof. In yet another embodiment, the iron chelator is selected from the group consisting of EDTA (ethyl enediaminetetraacetic acid), DTPA (diethylene triamine pentaacetic acid), NTA (nitrilotriacetic acid), detoxamin, deferoxamine, deferiprone, deferasirox, glutathione, metalloprotein, ferrochel (bis-glycinate chelate), ceruloplasmin, penicillamine, cuprizone, trientine, ferrulic acid, zinc acetate, lipocalin 2, and dimercaprol.

According to one embodiment of the invention, the components of the redoxome particles are natural chelators and approved diet supplements. The natural chelators of the invention are, without limitation, citric acid, amino acids (e.g. carnosine), proteins, polysaccharides, nucleic acids, glutamic acid, histidine, organic di-acids, polypeptides, phytochelatin, hemoglobin, chlorophyll, humic acid, phosphonates, and transferrin. In one embodiment, the chelator belongs to the group of polyphenols, such as flavones and flavonoids. In one embodiment, the polyphenol is, without limitation, rutin, quercetin, lutein, and EGCG.

According to one embodiment, the redoxome particle may be loaded with such antioxidants as phenol antioxidants such as dibutylhydroxytoluene (BHT) (IUPAC name: 2,6-bis(1,1-dimethylethyl)-4-methylphenol); dibutylated hydroxyanisole (BHA); propyl gallate; sodium sulfate; citric acid; sodium metabusulfite; ascorbic acid; tocopherol; tocopherol ester derivatives; 2-mercaptobenzimidazole or a combination thereof. In one embodiment, the amount of an antioxidant to be used is from 0.1 to 20% by mass. In yet another embodiment, the amount of an antioxidant to be used is from 0.5% to 10%; 0.7% to 100; 10 to 80; 3% to 80; 20 to 50; 5% to 100; 30 to 10%; and 2.5% to 10% by mass per the total mass of the film dosage composition. In one embodiment, the redoxome particles are enriched with LC-PUFA, docosahexaenoic acid (DHA) or ethanolamine plasmalogens or derivatives thereof. In yet further embodiment, redoxome particles deliver DFX which inhibits age-mediated collagen fragmentation. In one embodiment, the redoxome particles deliver ascorbic acid or derivatives thereof which inhibit hyaluronic acid degradation, to thereby delay collagen and extracellular matrix loss. In one embodiment of the invention, the redoxome particles may be useful in wound healing; aesthetic medicine; and adjunctive to dermal filler procedures. In one embodiment of the invention, the redoxome is derived from human foreskin/dermal fibroblasts, keratinocytes, adipose derived stem cells, and skin microbiome cells.

According to the embodiments of the invention, bioxome particles are categorized according to the cargo or physical attributes. In one embodiment, the bioxome particle is pH-sensitive bioxomes. In one embodiment, the bioxome particle is nucleic acid transfection bioxomes. As used herein, the term “nucleic acid transfection bioxome” refers, without limitation to nucleotides encapsulated by the bioxome and delivered to the target cell, tissue, organ for the purpose of modulating the expression of a target polynucleotide or polypeptide. In the context of the invention, the term “modulating” refers, without limitation to increasing; enhancing; decreasing; eliminating the expression of an endogenous nucleic acid or gene or of a corresponding protein. In one embodiment, such encapsulated polynucleotides may be natural or recombinant in nature and may exert their therapeutic activity using either sense or antisense mechanisms of action. In one embodiment, the nucleic acid transfection bioxome is supplemented with synthetic cationic lipids. In another embodiment, the bioxome particle is long circulating, slow release bioxome. The phrase “long circulating, slow release bioxome” refers to bioxome engineered to provide sustained delivery of the cargo by bioxome core polymer or protein or polysaccharide modification, and to therefore prolong therapeutic level of drug in the blood circulation or target tissue. The long circulating, slow release bioxome of the invention is manufactured, without limitation, by addition of core-PEG conjugated lipid, albumin, Hyaluronic Acid; anchoring metal ions; or a combination thereof. Hydrophilic modification of the core bioxome membrane increases residence time of the bioxome in the target organ or blood in comparison with the non-modified bioxome. The prolonged circulation time is attributed to the decreased rate of the absorption of the plasma protein on the surface of the PEGylated particle.

In one embodiment, the bioxome particles are selective targeting bioxomes. In the context of the invention, the term “selective targeting bioxome” refers, without limitation, to bioxome particles designed for specific targeting ligand or homing moieties. In the selective targeting bioxome of the invention, the ligand or homing moieties are, without limitation, glycosaminoglycan; monospecific or bispecific antibodies; aptamers; receptors; fusion proteins; fusion peptides; or synthetic mimetics thereof; cancer targeting-folic acid; specific phospholipids; cytokines, growth factors; or a combination thereof.

In yet further embodiment, the bioxome particle is immunogenic bioxome. In the context of the invention, the term “immunogenic bioxome” refers to bioxome particles derived from pathogen cell culture, or bioxome particle with co-extracted, or externally embedded immunogenic moieties. As used herein, the term “immunogenic” refers, without limitation to the ability of a particular substance, such as an antigen or epitope, to provoke an immune response in the body of a human and other animal. In other words, immunogenicity is the ability to induce a humoral and/or cell-mediated immune responses.

In one embodiment, the membrane of bioxome particles of the invention comprises at least 50% from cell membrane obtained from the cellular source cultured in pre-defined cell culture conditions. In one embodiment, the bioxome particles derived from different sources may show differences in lipid composition compared to the plasma membrane.

The invention further provides a composition comprising the bioxome particle and at least one carrier. In one embodiment the composition is a pharmaceutical composition and the carrier is a pharmaceutically acceptable carrier. In one embodiment, the composition is suitable for oral, intravenous, subcutaneous, intraperitoneal, intra-muscular, topical, ocular, intra-nasal, including directly to olfactory bulb for CNS delivery, rectal, vaginal, pulmonary, sublingual, transmucosal, intra-tissue, by ultrasound guided, endoscopic, through tissue inserted implant administration, intrathecal, and transdermal administration. In yet another embodiment, the composition is a cosmeceutical composition and the carrier is a cosmeceutically acceptable carrier. In yet another embodiment, the carrier a food grade carrier. In one embodiment, the composition is an edible composition the carrier is food grade carrier. In one embodiment, the composition further comprises excipients, safety tested active compounds, reagents, or a combination thereof. In one embodiment, the concentration of the bioxome particles in the composition is 0.005% to 80% of the total composition. In yet another embodiment the concentration is 0.005% to 25%. In one embodiment, the bioxome particles are formulated with suitable excipients and carriers; packaged and stored for cell banking purposes, cell therapy purposes, imaging or drug delivery purposes, as reagents for transfection or reagents for research kits. The composition comprising the bioxome particles are, without limitation, solid, liquid, semisolid, cryopreserved, refrigerated or dried ready-to-use.

The invention further provides a process for the manufacture of a sample comprising a plurality of bioxome particles, wherein the bioxome particles are engineered to carry a cargo comprising at least one active molecule and designed to undergo fusion with a target cell to release the cargo; and wherein said bioxome particles comprise a cell membrane component derived from a selected cellular or extracellular source; the process comprising:
a. Performing total cell lipid extraction from the selected cellular or extracellular source in a mild solvent system to obtain a lipid extract;
b. Drying the lipid extract; and
c. Inducing self-assembly of bioxome particles by performing at least one step of ultra-sonication;
   wherein the resulting bioxome particles in the sample are characterized by an average particle size of 0.03 μm to 5 μm. As used herein, the term “mild solvent” refers, without limitation, to any of solvents of Class3 or of Class 2 with PDE>2.5 mg/day and Concentration limit>250 ppm in accordance to
   https://www.fda.gov/downloads/drugs/guidances/ucm073395.pdf

In one embodiment, the average particle size is 0.05 μm to 3 μm. In yet another embodiment, the average particle size is 0.08 μm to 1.5 μm. In further embodiment the average particle size is 0.1-0.7 μm; 0.1-0.5 μm, 0.2-0.5 μm; 0.3-0.5 μm. In another embodiment, the average particle size is 5 μm or less; 1.5 μm or less; 0.7 μm or less; 0.5 μm or less; 0.3 μm or less; 0.15 μm or less. In one embodiment, the average particle size is 0.5 μm to 1.5 μm. In one embodiment, the average particle size is 0.4 μm to 0.8 μm. In another embodiment, the average particle size is 0.3 μm to 0.5 μm. In one embodiment, the sample comprising the bioxome particle has the pH of 4.5 to 5. In yet another embodiment, the sample comprising the bioxome particle has the pH of 4.5 to 5. In one embodiment, the solvent system comprises a mixture of polar and non-polar solvents. In one embodiment, the polar solvent in the solvent system is selected from the group consisting of isopropanol, ethanol, n-butanol, and water-saturated n-butanol. In one embodiment, the non-polar solvent in the solvent system is selected from hexane and solvents from the terpene group. In one embodiment, the non-polar solvent in the solvent system is n-hexane. In one embodiment, hexane may be fully or partially suspended by supercritical fluid extraction using supercritical carbon dioxide (scCO²) as a mild “green” solvent has many advantageous properties, including gas-like viscosity, liquid-like density, about 100-fold faster diffusivity than in organic solvents at ambient conditions, as well as operation at relatively low temperature. Terpene/flavonoid may be selected further from alpha-pinene, d-limonene, linalool, eucalyptol, terpineol-4-ol, p-cymene, borneol, delta-3-carene, beta-sitosterol, beta-myrcene, beta-caryophyllene, cannflavin A, apigenin, quercetin and pulegone. In one embodiment, the solvent from the terpene group is selected from the group consisting of d-limonene, α-pinene and para-cymene.

In one embodiment, the polar solvent in the solvent system is isopropanol, and the non-polar solvent is n-hexane. In yet another embodiment, the solvent is Hexane-Isopropanol 3:2 low toxicity solvent mixture. in one embodiment, the the solvent system further comprises a stabilizer. In another embodiment, the stabilizer is butyl-hydroxytoluene (BHT). In one embodiment, the solvent system may further comprise additives such as, without limitation, antioxidants, surfactants, stabilizers, vitamin E, squalene, and cholesterol, or a combination thereof. In one embodiment, the process further comprises the step of co-precipitation of a nucleic acid. In one embodiment, the nucleic acid is ribonucleic acid (RNA) or deoxyribonucleic acid (DNA). In one embodiment, the nucleic acid is RNA. RNA delivery is one of the major challenges. In one embodiment, the bioxome engineering is achieved using cell membrane collected from cellular or extracellular source through hydrophilic-hydrophobic self-assembly during cavitation ultrasonication procedure in hydrophilic vehicle. In one embodiment, bioxome particles are extruded after lipid membrane isolation post ultrasonication. In one embodiment, the cargo comprising the active molecules is hydrophilic, and is entrapped into hydrophilic vehicle during ultrasonication, or during extrusion. In yet another embodiment, the cargo is hydrophobic cargo and is entrapped prior to extraction with the solvent system, during extraction, during drying/solvent evaporation procedure, during ultrasonication, during extrusion. Repetitive freeze thawing may improve rate of encapsulation of hydrophilic cargo post drying and post ultrasonication. The level of encapsulation loading is affected by selection of engineering parameter based on sensitivity, stability and desired loading dose of selected cargo as predesigned at each specific therapeutic or research moiety. In one embodiment, the active molecule may be interwoven into Bioxome core at predefined concentration without risk for viral gene vectors impurities as safety concerns. In one embodiment, the bioxome particles may be electroporated or microinjected. In one embodiment, RNA or DNA may be incorporated into the bioxome particles through gentle ultrasonication at 4° C. in the presence of any suitable protective buffers to maintain integrity of nucleic material for therapeutic delivery. According to the embodiments of the invention, the manufacturing process is compliant with most known industrial features of LNPs and liposomes.

In one embodiment, the cellular or extracellular source for total lipid extraction is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, postnatal umbilical cord, placenta, amniotic sac, kidney tissue, neurological tissue, biological fluids, and excrement or surgery extracted tissues, (i.e. milk, saliva, mucus, blood plasma, urine, feces, amniotic fluids, sebum,), postnatal umbilical cord, placenta, amniotic sac, kidney tissue, neurological tissue, adrenal gland tissue, mucosal epithelium, smooth muscle tissue, adrenal gland tissue, mucosal epithelium, smooth muscle tissue, a bacterial cell, a bacterial culture, a whole microorganism, conditional medium, amniotic fluid, lipoaspirate, liposuction byproducts, and a plant tissue. In yet another embodiment, the lipid extraction is performed from cell-conditioned media, lyophilized conditioned cell media, cell pellet, frozen cells, dry cells, washed cell bulk, non-adhesive cell suspension, and adhesive cell layer. In yet another embodiment, the cell layer is grown in cell culture plasticware coated or uncoated by extracellular matrix or synthetic matrix, selected from a (multi) flask, a dish, a scaffold, beads, and a bioreactor. According to one embodiment of the invention, the membrane extract is dried by freeze or/and spray/freeze drying. In yet another embodiment, the membrane extract is dried by evaporation. The evaporation can be carried out by any suitable technique, including, but not limited to speedvac centrifuge, argon/nitrogen blowdown, spiral air flow and other available solvent evaporation methods in controlled temperature environment, such as microwave or rotor evaporation, Soxhlet extraction apparatus, centrifuge evaporators. In yet further embodiment, the membrane extract is ultra-sonicated by tip ultra-sonicator in a buffer loaded with desirable active molecules. In one embodiment, the average particle size is 0.4 μm to 1.5 μm when particle size is measured within few hours after the preparation. In yet another embodiment, the particle size is 0.8 μm to 5 μm when particle size is measured within a month after the preparation and bioxome particles are stored at 0° C. to −4° C.

In one embodiment, the bioxome particles are derived from membranes of cellular or extracellular source. In one embodiment, the bioxome particles are engineered on-demand from a pre-defined source. In one embodiment, the cell-source is autologous. The term “autologous” refers to a situation when the donor and the recipient are the same. In one embodiment, the cell-source is non-autologous. In one embodiment the donor source is mesoderm cells including, but not limited to fibroblasts, mesenchymal stem cells, pluripotent and differentiated stem cells, cells of the immune system, dendritic cells, ectoderm, keratinocytes, cells of GI and oral cavity, nasal mucosal cells, neuronal and retinal cells, endothelial cells, cardiospheres, cardiomyocytes, pericytes, and blood cells. In one embodiment, the source for the bioxome particles is stromal cells, keratinocytes, melanocytes, parenchymal cells, mesenchymal stem cells (lineage committed or uncommitted progenitor cells), liver reserve cells, neural stem cells, pancreatic stem cells, and/or embryonic stem cells, bone marrow, skin, liver tissue, pancreas, kidney tissue, neurological tissue, adrenal gland, mucosal epithelium, and smooth muscle.

In the process of the invention, the bioxome particles can be loaded with selected active molecules. In one embodiment, the loading is performed during extraction. In yet another embodiment, the loading is performed during drying, prior to extraction or post. In one embodiment, the obtained bioxome particles may undergo extrusion.

The advantage of the HIP extraction system of the invention is that in contrast to classic chloroform-methanol lipid extraction, enables extract membrane lipids with minimal lipase activity and directly from/on chloroform-soluble components, such as plastics, cell culture sterile surface wells, including but not limited to hollow fiber, beads, nucleopore, and polycarbonate filters. For example, HIP would permit direct extraction from polycarbonate is stable in these solvents. HIP extraction can be used for consolation of Bioxomes from cells or conditioned medium in parallel with coextraction of RNA or proteins from same cell culture or tissue sample. For such process to cell layer or cell pellet or lyophilized conditioned medium or tissue extract HIP can be premixed with approx ¼-⅕ th per volume of water buffer or RNA or DNA or protein stabilizing solution (e.g. RNAsave bioind1.com or Trhaloze or RNAse inhibitor containing buffer). The water phase buffer or stabilizing solution extracts coprecipitated nucleic or protein extract wherein said coextracted nucleic or protein phase then may be separated for example by centrifugation or freezing gradient etc. Such RNA or/and DNA or/and protein containing phase may be further during particle formation with hydrophobic phase of bioxome particle and then used as biotherapeutics or for biomarker diagnostic or research reagent use.

In one embodiment, the process of the invention is compatible with GMP and GLP guidance. In one embodiment, according to the process of the invention, the bioxome particles are harvested from cell biomass; cellular pellet; adhesive cellular layer; medium; or a combination thereof. In one embodiment, the bioxome particles are extracted by single low-toxicity step that allow OECD approved-solvent extraction process.

In one embodiment, source cells can be modified prior to the extraction by exposure to mild oxidative stress, starvation, radiation or other in vitro modification of cells in culture, in culture to express more lipophilic antioxidants. In one embodiment the lipophilic anti-oxidant is rutin, squalene, tocopherol, retinol, folic acid and derivatives thereof. In one embodiment, the lipid solution component is filter-sterilized. In yet further embodiment, the lipid solution component can be stored in nitrogen or argon at a temperature of −20° C. to −80° C.

In further embodiment, the solvent further comprises detergent surfactant. In one embodiment, the detergent is Polaxomer. In one embodiment, the process comprises lyophilizing/evaporating HIP solvent portion to form a bioxome particle-nucleic acid complex; and ultrasonicating in a hydrophilic carrier/buffer, and/or optional extrusion with desired particle size.

The invention further provides a sample comprising a plurality of bioxome particles, prepared according to the process of the invention.

The invention further provides a method of treating or preventing a pathology in a subject in need of such treatment, comprising administering to the subject the pharmaceutical composition comprising the bioxome particle and at least one carrier. In one embodiment the composition is a pharmaceutical composition and the carrier is a pharmaceutically acceptable carrier. In one embodiment, the composition is suitable for oral, intravenous, subcutaneous, intraperitoneal, intra-muscular, topical, ocular, nasal, rectal, vaginal, pulmonary, sublingual, transmucosal and transdermal administration. In yet another embodiment, the composition is a cosmeceutical composition and the carrier is a cosmeceutically acceptable carrier. In one embodiment, the pathology is selected from the group consisting of an inflammatory disorder, a neurological disorder, an infectious disorder, a malignancy, a disorder of the immune system, and an autoimmune disorder. The non-limiting list of disorders comprises inflammatory disorders such as a chronic or acute inflammatory skin disorder, inflammatory bowel disease (IBD), arthritis, inflammatory respiratory disorder, or acute or chronic wound or injury, infectious disorder is an infectious disease caused by a bacterial pathogen, a viral pathogen, or a parasite; proliferative disorder is a malignancy associated with elevated level/s of pro-inflammatory cytokines and/or reduced level/s of anti-inflammatory cytokines; a metabolic disorder, diabetes mellitus type I, diabetes mellitus type II, or a diabetes related condition; inflammation mediated by elevation of pro-inflammatory cytokines, such as, without limitation, IL-1 alpha, IL-6, TNF-alpha, IL-17; inflammation mediated by elevating the level of at least one anti-inflammatory cytokine, such as, without limitation, IL-13, IL-4, or IL-10; chronic rhinosinusitis (CRS), allergic rhinitis; COPD; nasal polyposis (NP); vasomotor rhinitis, airways hyper-responsiveness, cystic fibrosis; lung fibrosis; allergic sinusitis, IBD; Crohn's disease; and ulcerative colitis.

In the context of the invention, bioxome particles can be incorporated into a broad range of aesthetic medicine, cosmetic, topical dosage forms including but not limited to gels, oils, dermal fillers, emulsions and the like. For instance, the suspension containing the particles can be formulated and administered as topical creams, pastes, ointments, gels, lotions and the like. In certain embodiment, the aesthetic medicine application may be made by topical, “open” or “closed” procedures. By “topical,” is meant the direct application of the pharmaceutical composition to a tissue exposed to the environment, such as the skin. “Open” procedures are those procedures include incising the skin of a patient and directly visualizing the underlying tissue to which the pharmaceutical compositions are applied. “Closed” procedures are invasive procedures in which the internal target tissue instruments into site of disease directly, for example in conjunction with dermal filler during aesthetic procedure, or upon! local topical in the skin areas, wounds, wrinkles. The invention further provides a method of improving a skin condition in a subject in need comprising administering to the subject the composition comprising bioxome particles of the invention. In one embodiment, the invention further provides use of bioxome particle as delivery vehicles for active-biomolecules. Active biomolecules of the invention have, without limitation, anti-inflammatory activity; anti-aging activity; anti-cancer activity; metabolic activity. or genetic DNA or RNA bioactive material. In one embodiment, the active biomolecules are cellular cytokines. In yet further embodiment, the active biomolecules are growth factors. In one embodiment, the active biomolecules are oligonucleotides, comprising either deoxyribonucleic acids or ribonucleic acids. In one embodiment, the length of the oligonucleotide is 2 to 100 nucleotides. In one embodiment the oligonucleotide is natural, synthetic, modified or unmodified. In one embodiment, the oligonucleotide is RNAi; siRNA; miRNA mimetics; anti-miR; ribozymes, aptamers, exon skipping molecules, synthetic mRNA, short hairpin RNA (shRNA). In yet further embodiment, the membrane of the bioxome particle is engineered to provide controlled release of the cargo to the target. In one embodiment, oxidative stress-sensitive lipids are embedded in the membrane of bioxome particles. In another embodiment, the bioxome particles are loaded with metal chelators and antioxidants. In one embodiment the exosomes-like particles are used to introduce pluripotency factors into somatic or stem cells to thereby obtain non-viral induced pluripotent stem cells-iPSC. In yet another embodiment the bioxome particles are used to treat cells. Treatment of the cells with the bioxome particles of the invention is carried out at physiological temperatures (about 37° C.). Duration of treatment is 2 min to 72 hours. The invention further provides the bioxome particles of the invention for use as a medicament.

In the embodiments of the invention, the QC specifications for particle size characterization of bioxome particles include, without limitation, the following: particle size; penetration capacity to the target tissues/cells; sterility; non-immunogenicity and safety defined by absence of proteins and nucleic acids. Particle size distribution is measured on Malvern Nano Zetasizer and refined by Zetasizer software. The size of the bioxome particles assemblies are manipulated based on the desired application, making use of commonly available down-sizing techniques. The assemblies may be down-sized by extrusion through membranes with preselected mesh dimensions.

In the context of the invention, the QC specifications for bioxome particles lipid characterization include, without limitation, the following: bioxome particles are qualified and quantified by membrane lipid composition and characteristics, such as: (1) de/saturation index of fatty acids-FA, (2) FA chain length characteristics, (GC; HPLC analytical methods) i.e. Long chain LC-polyunstarurated FA PUFA/medium chain-MC/; (3) polarity (IZON assay); (4) lipid composition, i.e. Content percentage ad/or ratio, e.g. PL-phospholipid composition and ration PC-PE/PI-PS or ratio/percentage between various lipid groups of the Bioxome membranes, e.g. PL/NL (neutral lipid)/CL/GL/TG/FFA (HPLC; TLC; LC-MS; MALDI; column chromatography; etc.); (5) total lipid (vanillin assay, etc.); (6) optional functional lipids and lipid derivatives content, e.g. prostaglandins, prostacyclines, leukotriens, tromboxanes (HPLC; MS-MS; ELISA; RIA; etc.), or (7) metabolites such as hydroxy index- (iodine assay); and (8) ROS mediated oxidation.

In the context of the invention, the QC specifications for final composition comprising bioxome particles include, without limitation, the following: viscosity and osmolarity; pH; number of particles per batch; turbidity; stability specification parameters. Methods of particle measurements and characterization that are provided by IZON Ltd., are also applicable for QC in bioxome particles production.

In the context of the invention, the QC specifications for the bioxome production potency include, without limitation an assay for desired bioxomes activity. For example, cell culture assay to test bioxome and redoxome based products functional effect in vitro. The effect may be screened as QC potency assay by scratch assay, cytotoxicity assay, for example chemotherapeutic drug cytotoxicity assay, ROS generating or hydroxyurea aging inducing assay, inflammation IL19 or TGF beta inducing assay.

### EXAMPLES

### Example 1. Lipid Extraction by Solvent from Cell Pellets

The following cell samples were cultured: Sample 1—Primary Umbilical Vein Endothelial Cells; Normal, Human (HUVEC) (ATCC® PCS-100-010™); Sample 2—Primary Mammary Epithelial Cells; Normal, Human ATCC® PCS-600-010™; Sample 3—Human Foreskin Fibroblasts HFF (primary-donated); Sample 4—Human Adipose-Derived Mesenchymal Stem Cells; (ATCC® PCS-500-011). All cell samples for were passaged 3-6 passages, each sample was stored with 2×10upp⁶ cells per cryopreservation vial and pellets were collected and stored in cryopreservation medium (GIBCO) in liquid nitrogen. The pellet was washed with PBS and cellular mass was concentrated by centrifugation. Cell concentrate was mixed with HIP solvent mixture composed of Hexane:Isopropanol (3:2) solvents and 0.02% BHT. The pellets were repeatedly vortexed at room temperature for 2-4 minutes per vortex cycle. The samples were then centrifuged and the supernatant was collected. The collected supernatant was subjected to evaporation until an oily residue was formed. Bioxome particles were further prepared and encapsulated.

### Example 2. Particle Formation and Characterization

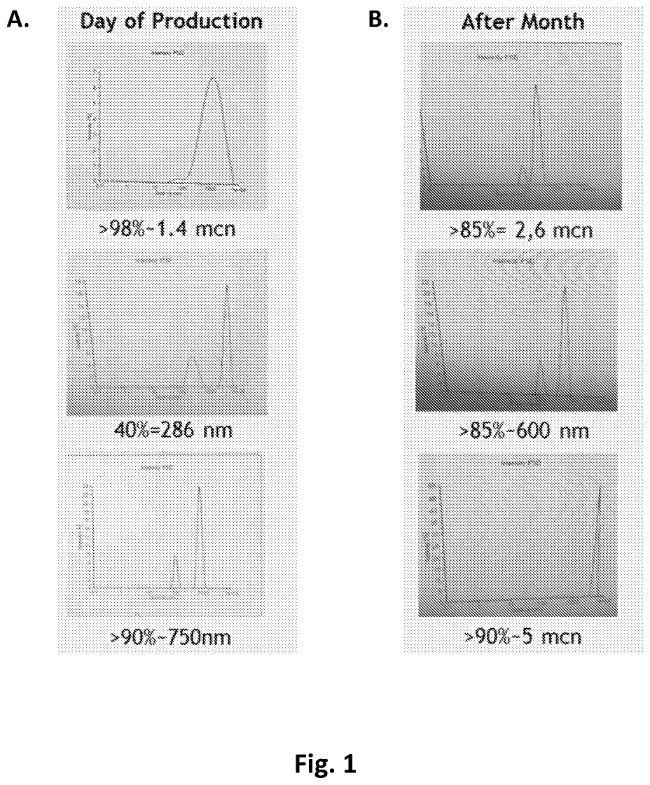

Bioxome particles formation was performed using tip ultra-sonication (vibra-cell Sonics): 3 sonication cycles per sample, each cycle 3 seconds with 10 seconds interval between cycles. During the procedure samples maintained at ambient temperature by cooling sample with ice during the sonication (Sonicator temperature monitored at 60° C.) in order to prevent heating induced by sonication. pH of the final product was measured and particle size was identified using Nanosizer-Malvern as illustrated by FIG. 1A. The properties of formed product fell into the QC specifications of pH measurement of 4.5-5, and average particle size of size 0.05-1.5 μm. For stability testing, the obtained samples were stored for a month at 4° C. FIG. 1B. The observed average particle size measured using Nanosizer-Malvern was significantly larger, indicating aggregation/interfusion of Bioxome particles.

### Example 3. Establishment of an Experimental System Designed to Produce Bioxomes

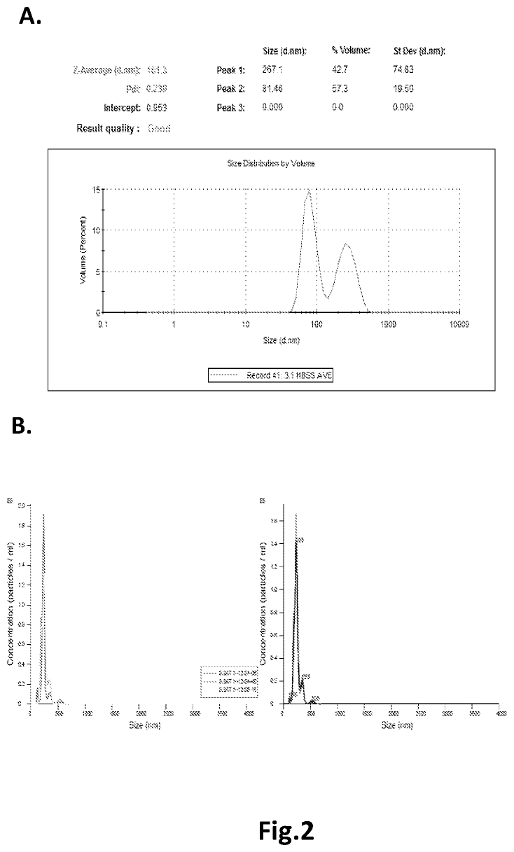

Protocols for bioxome production from lipids extracted by solvents were established using a frozen cell pellet or fresh culture of the same source of cells. The cells used for protocol establishment were human mesenchymal stem cells (MSCs) derived from adipose tissue; bone marrow cells, and HepG2 liver cell line. Lipid membranes were extracted using Hexane:Isopropanol solvents and dried by a nitrogen gas evaporation or lyophilizer. Fresh cell cultures were cultured with HBSS-HEPES to generate condition media for 2 h prior to lipid extraction. The resulting condition media was also collected, and lipids and RNA were extracted and dried. The dried-lipid samples produced from cell pellets or cell cultures were ultrasonicated in HBSS to produce bioxomes. The concentration of the RNA extracted from each sample (using DDW) was measured using a NanoDrop Lite spectrophotometer. Formed bioxomes were evaluated for their size and stability using the ZetaSizer nano (Malvern Panalytical Ltd.) particle size analyzer and the NanoSight analyzer (Malvern Panalytical Ltd.). The measurements using the ZetaSizer nano showed two particles size distributions with peaks at 81 nm and 267 nm as represented in FIG. 2A. The NanoSight results indicated an average particle size of 248 nm FIG. 2B.

### Example 3. Examination of Bioxome Stability

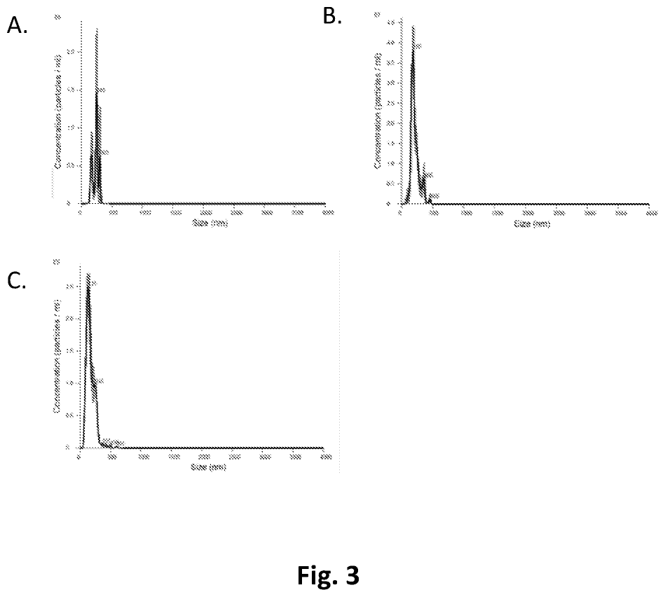

For the examination of bioxome stability, a sample of bioxomes produced by sonication from bone marrow hMSC described in Example 2, was split into 8 sub-samples following bioxome production. Each pair of samples were exposed to different number of freeze-thaw cycles (0-3 cycles). Half of the samples (one of each pair) were lyophilized, the rest were stored in −80° C. freezer. Measurement of the bioxome particle size using the NanoSight indicated that all samples are stable regardless of the number of freeze-thaw cycles, however the uniformity of the particles size is condition dependent FIG. 3. The results revealed that an addition of ultrasonication step (with or without lyophilizing) following freeze-thaw cycles, improved the uniformity of the bioxome size FIG. 2A-C.

### Example 4. Bioxome Fusion Experiments

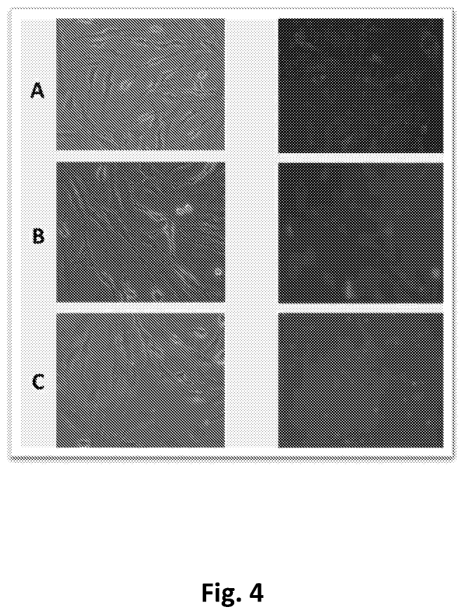

Bioxome particles were loaded with fluorescent lipid Biomarker (BioDipy™ TR (D7540), ThermoFisher Scientific) to mimic active hydrophobic compound and visualize Bioxome inter- and intra-cellular trafficking. The BioDipy™ and NBD fluorophores incorporated in fluorescent sphingolipids have different spectroscopic properties. The BioDipy™ FL fluorophore produces greater fluorescence output than NBD due to its high molar absorptivity and fluorescence quantum yield. It is also more photo-stable than NBD. The NBD-labeled sphingolipids have higher rates of transfer through aqueous phases than their BODIPY FL counterpart. FIG. 4 demonstrates fusion of fluorescent Bioxome particles. Final concentration of 5 μM of BioDipy was fused into membrane of confluent Human Foreskin Fibroblasts (HFF) cells. Fluorescence was visualized using fluorescent microscope (at ×40 magnification) and CDD camera and the images were recorded. Fusion of fluorescent bioxome particles to HFF cells was tracked during 10-35 min per sample. Between measurements, cells were kept in 37° C. All bioxome preparations (example 1, samples 1 to 4) fused into target cells within 15 min., FIG. 4A-C.

### Example 5. Cell Membrane Extraction by HIP System from Adherent Cell Layer in Conditioned Medium

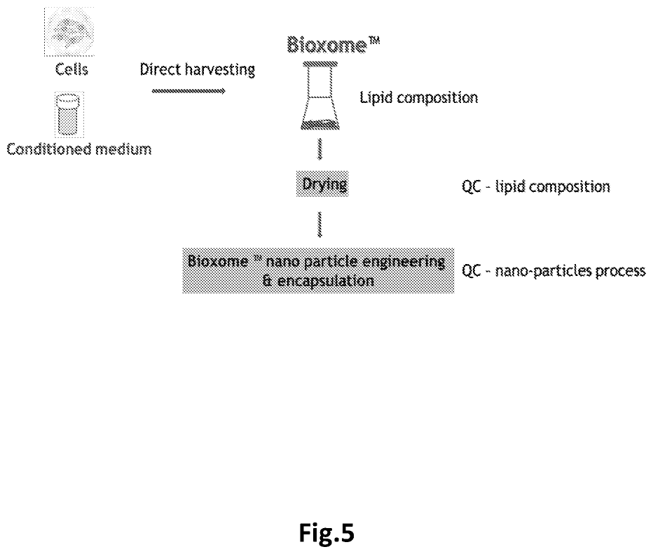

Extraction Human Foreskin Fibroblasts (HFF) cells and conditioned medium were separated as shown in FIG. 5. Adherent HFF cell layer was washed four times from growth medium with HBSS+HEPES. The buffer was then removed and HIP solvent system including 0.02% BHT was added to cover the cell layer in the multi-flask. Extraction was performed by gentle shaking of the flask at ambient temperature during 20 min. The contents of the flask were transferred into 50 ml culture tubes and water (36° C.) evaporation under Argon was performed resulting in formation of dry, oily film-like residual.

### Example 6. Engineering of Bioxome Particles with Membrane Proteins

Cells were seeded and collected according to Example 1. Extraction of the lipids and membrane proteins from the cell mass was performed in mild solvent mixture comprising 1:1 mixture of Hexane Isopropanol. Following the extraction, supernatant and the pellet were collected, and total protein was measured by Bradford assay with BSA standard. Protein content of 15% was measured. Nucleic acids were removed from the crude extract by washing with NaCl and a measurement by spectrophotometer at 280 nm was performed.

### Example 6. Preparation of Exosome-Like Particles Encapsulated with Oligonucleotide

Cells are seeded and collected according to example 1. The cell mass is dissolved in HIP (3:2 v/v) solvent system or hexane/ethanol (2:1 v/v) solvent system. After 30 min. incubation at room temperature, further solvent extraction step (about ¼ of the original volume each) is performed. The extract is then centrifuged at 3000 rpm for 10 min. using a bench type centrifuge and a clear interface between the aqueous and the solvent phase is formed. The oligonucleotide is solubilized in an aqueous solution matching that of the extruded vesicles (pH 4, 30% ethanol) and is added drop-wise to the HIP extract of cell membrane. The solvent phase is subjected to gentle N₂ blow to remove the solvent. The sample is then placed in SpeedVac for 3 hours to evaporate residual solvent. The membrane-nucleic acid containing vessels are filled with a HBSS buffer (20 mM HEPES, 150 mM NaCl, pH 7.2) and ultra-sonicated.

### Example 7. Dynamics of the Delivery of Fibroblast-Derived Bioxome Particles to Fibroblasts Cell Target Vs. Glial Cell Target

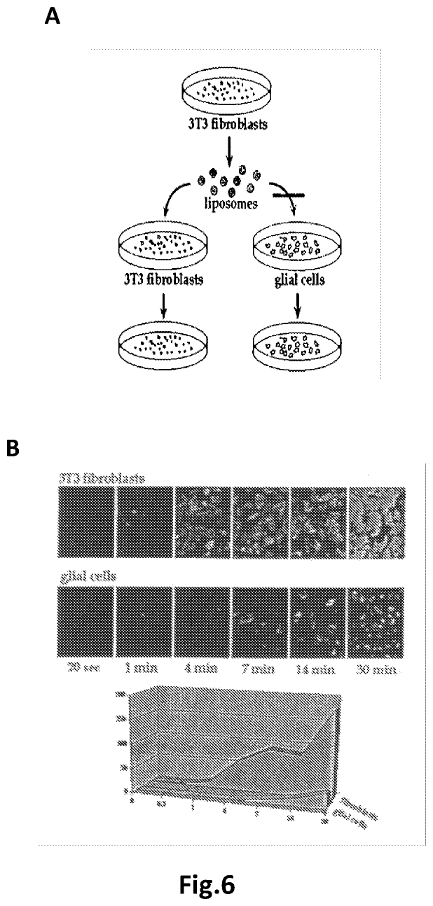

3T3 NIH fibroblasts were pre-labeled with fluorescent C6-nbderythro-ceramide for 15 min at 37° C. (final concentration of C6-nbderythro-ceramide was 5 μM). Medium was discarded, dishes were subsequently washed twice with ice cold PBS. For the extractions, cells were incubated with 1 ml Hexane:Isopropanol (HIP) 2:1 (v/v) for 15 min at RT during shaking. HIP was collected from 3 Petri dishes into one glass tube. Dishes were washed once with 1 ml HIP each, adding all washes to the tube. HIP was evaporated under the stream of N₂. After re-suspending in 300 μl PBS, the cell mass was sonicated. For analysis of NBD label release and incorporation, NIH 3T3 fibroblasts and primary cultured glial cells were seeded into 13 mm glass cover slips in 24 well culture dish one day prior to the experiment. Bioxome particles prepared from NIH 3T3 fibroblasts were added to the cells (10 μl of the bioxome suspension to each well), cover slips were removed from wells at indicated time intervals, washed to remove unbound NBD-label, and were subjected to analysis using fluorescent Zeist microscope (at ×40 magnification) and CDD camera. Images were recorded in the computer. Scheme for general procedure of this experiment is presented in FIG. 6A.

### Results:

Results of imaging are presented in FIG. 6B. Significant labeling of the fibroblast cells by the labeled bioxome particles was observed within 4 min. This signal became even more intense 30 min after applying the bioxome particles. In contrast, glial cell showed almost no binding of the labeled bioxome particles, even after 30 min. These results indicate specific targeting of the bioxome particles that were originated from fibroblasts, to the fibroblast target cells.

### Example 8. Engineering of Redoxomes

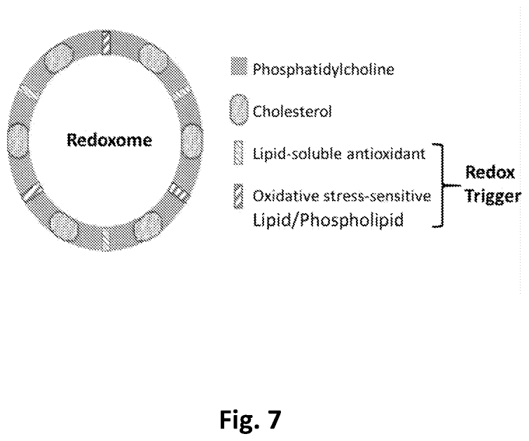

Schematic representation of redoxome particle is demonstrated in FIG. 7. Lyophilized cells of Lactobacillus bacteria were used as a source for bioxome extraction. Tocopherol and cholesterol (0.5%) were dissolved in a solvent solution consisting of Hexane:Isopropanol (HIP) (3:2 v/v) with 1.5% butyl-hydroxytoluene (BHT) as a stabilizer. To increase stability/rigidity of the lipid bilayer membrane, the lipophilic antioxidants alpha-tocopherol (˜3%), DHA (˜3%), cholesterol as rigidity stabilizer (1.5%) were used. The solvents were evaporated under a stream of nitrogen, and lyophilized for 2 hours at 4° C. The resulting lipid dispersion was sonicated for 10-20 min in a tip-type ultra-sonicator until the turbidity had cleared. Electron paramagnetic resonance (EPR) spin trapping was used to detect lipid-derived free radicals generated by iron-induced oxidative stress in exosome-like particles. Using the nitrone spin trap a-(4-pyridyl-1-oxide)-N-tert-butylnitrone (POBN), carbon-centered radical adducts were detected. Ascorbic acid and DFX or EDTA were added to the buffer during ultra-sonication. Extracting desired membrane material from a cell culture selected source was carried out with an extraction solvent mix comprising HIP that dissolved selected lipid-Redox active reagents (DHA/EPA—as Redox-sensor or/and alpha-tocopherol-as Redox stabilizer). Lipid extraction by HIP was carried out using the following procedure: following the evaporation step, the desired diameter of vesicles was achieved by tip ultra-sonication.

### Example 9: EPR-Redox Sensitivity of the Redoxome Particles

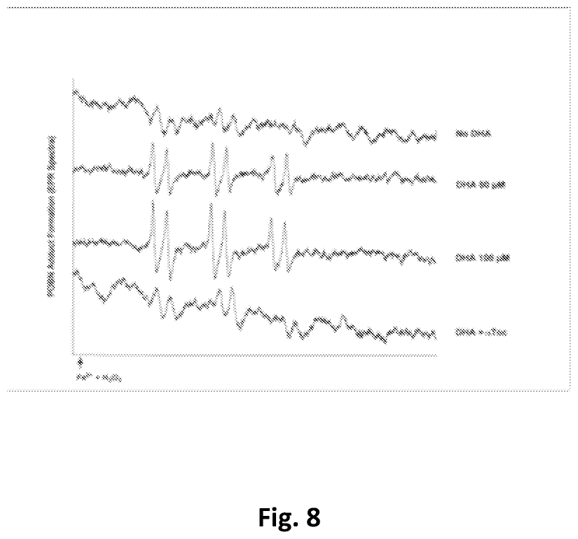

50 and 100 μM DHA was incorporated into the lipid bilayer membrane of redoxome particles FIG. 8, two middle spectra. A carbon-centered spin adduct was observed, with an increase in the spin adduct EPR signal intensity observed with increasing DHA concentration (indicating a DHA dose-dependent increase in LR formation). A very weak (control) spectrum was obtained FIG. 8 top spectrum in redoxome particles without DHA (representing basal POBN adduct formation). When the stabilizer alpha-tocopherol was incorporated into the DHA-enriched exosome-like particles, LR formation was observed to be reduced almost to basal levels FIG. 8 bottom spectrum, indicating an inhibition of LPO by the antioxidant incorporated in the exosome-like particles membrane.

### Example 10: Redoxome Particles Stability Under Normal Conditions

Calcein-containing redoxome particles were prepared by adding a self-quenching concentration of 60 mM calcein in 10 mM TRIS at pH8 (NaCl 100 mM) to the lyophilized material, followed by re-suspension in 0.2 ml buffer by vortexing. The non-encapsulated calcein was removed from the redoxome particle suspension by gel filtration, using a Sephadex G-50 column (Pharmacia). 30 μl of the redoxome suspension was injected onto the column and eluted in 10 mM TRIS at pH8 (NaCl 150 mM), and fractions of the eluent were collected. The fluorescence was monitored in untreated redoxome particles, as well as in redoxome particles exposed to the detergent Triton X-100 at the final concentration of 0.2%, by fluorescence spectroscopy at the excitation wavelength of 490 nm and emission wavelength of 520 nm.

### Results:

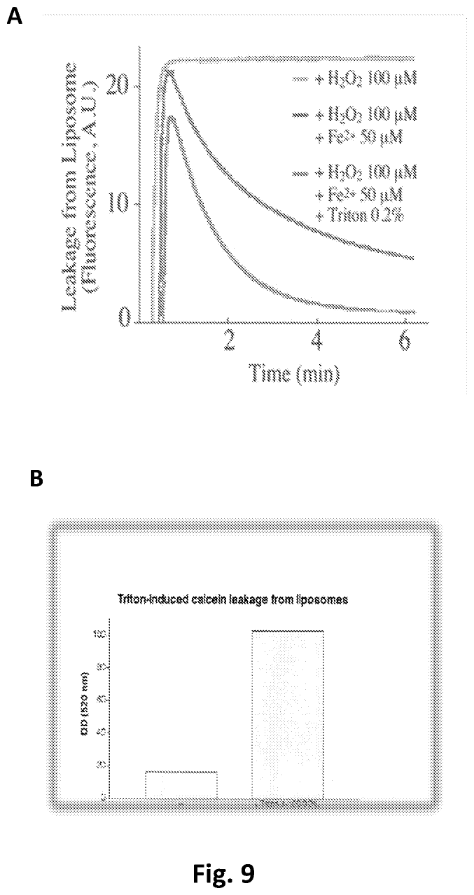

No change in calcein release upon addition of 100 μm H₂O₂ to the redoxome particles suspension was observed, FIG. 9A. Upon addition of 50 μm Fe²⁺ to the suspension, which acts as a catalyst to produce hydroxyl radicals from H₂O₂ via the Fenton reaction, a significant increase in the rate of change of fluorescence was observed in population of redoxome particles containing DHA or α-tocopherol, but not in the control population. When Triton X-100 was added to the solution FIG. 9B, all the calcein-remaining in the redoxome particles was released, with faster kinetics than the release observed upon addition of H₂O₂+Fe²⁺.

### Example 11: Effect of Redoxomes on Brain Damage

To test effect of Redoxomes on brain damage, mice (n=six per group) were sacrificed at postnatal age on Day 8 after birth, anesthetized by isoflurane and ketamine/pentobarbital sodium, in accordance with Helsinki compliance for animal studies. Then, freshly dissected brain slices subjected to Fenton reaction by incubation with 50 of ferrous sulfate heptahydrate (Sigma) for 15 minutes in 37° C. in DMEM plus Hepes. By such ex-vivo induced oxidative stress, brain slices were exposed to ROS production that mimic inflammatory diseases; ischemic stress; and other brain disorders, such as the Down Syndrome; atherosclerosis induced coronary ischemic alterations. TEARS released ROS generation into incubation medium by brain slices increased from −80-100 nM/mg wet weight to 120-160 nM per mg/wet weight. Treatment with DFX plus alfa tocopherol (vE) at concentration 1 μM, which was non-encapsulated in Redoxome, resulted in 20-30% reduction of TEARS release. When slices were co-incubated with hAdTMSC-Redoxomes encapsulated with 0.5 mcM DFX and vE—TBARS production was reduced by 50-60%.

### Example 12: In Vitro Potency Assay of Redoxomes

Neural Progenitor Cells Derived from XCL-1 DCXp-GFP (ATCC® ACS-5005), were incubated with ROS inducing Fenton reagents. For LPO measurement, as a selected marker of cell/tissue damage, to valuate feasibility of Redoxome treatment, the aliquots from hexane isopropanol—HIP (3/2 by volume) extracts were evaporated to dryness and dissolved in methanol for micro determination of lipid peroxides. Determination of aldehydic lipid peroxidation products: malonaldehyde and 4-hydroxynonenal. To 0.5 ml aliquots of the incubation medium, an equal volume of thiobarbituric acid (0.34% TBA in 50% acetic acid glacial) was added. After boiling for 10 minutes in water bath, the rose color developed with fluorescence at 535 nm excitation wavelength and 553-emission wavelength. An appropriate standard curve was run in parallel (1,1,3,3-tetraethoxypropane, Sigma). For measuring tissue levels of LPO after in vivo experiments, frozen tissue slices brain, liver, lung, skin, kidney, as relevant, stored at −70° C. after in-vivo experiment, were thawed in cold 4° C. PBS, on ice; rinsed once or twice depending on tissue source. The tissue extract was obtained by extraction with cold glacial 10% TCA containing 0.01% w/v butylated hydroxy toluene (BHT, Sigma). The tissue was further homogenized for 30 sec by high speed homogenizer on ice and then centrifuged for 10 minutes for 3500×g. Aliquots of the supernatant of tissue extracts were tested for LPO, measured as malondialdehyde products released to the supernatant following extraction. In tissue samples, LPO was represented as TBA—reactive substance, (TEARS) per wet weight.

### Results:

Basal levels of TEARS in various tissues were similar: in rat brain slices 40-50 pmol/g wet weight, in the liver −60-80 pmol/g wet weight. LPO release to the conditional medium increased 5 to 10-fold after ROS induction by Fenton reaction (ferrous sulfate heptahydrate (Sigma) 0.1 mM plus 0.2 mM H₂O₂ for 20 min) in iPSC neuronal precursors. Co-incubation with bone marrow Redoxomes containing ferrous sulfate heptahydrate (Sigma) inhibited LPO increase between 40-80%.

Treatment with Concovalin A induced approximately two-fold increase in LPO in liver, wherein concurrent treatment with Redoxome derived from human mesenchymal adipose tissue encapsulated with 5 mcM of DFX and 5 mcM alpha-Tocopherol reduced LPO to basal level.

### Example 13: RNA Encapsulation and Electroporation Experiments

### RBC:

Bioxomes were prepared from human red blood cells (RBC). RBCs were preferably collected from Group 0 blood samples, then were separated from plasma and white blood cells by centrifugation and leukodepletion filters (Terumo Japan). Bioxome extraction procedure was performed as described above. Electroporation experiments were performed using a Gene Pulser Xcell electroporator (BioRad), exponential program at a fixed capacitance of 100 μF with 0.4 cm cuvettes. E12 Bioxomes obtained from E9 RBCs were diluted in OptiMEM (ThermoFisher Scientific) and were mixed with 4 μg Dextran conjugated with AF647 (ThermoFisher Scientific) to a total volume of 2001 μl, 100 μl of Bioxomes aliquots added to each cuvette and incubated on ice for 15 min at 150-250V. In a case that aggregated formed, deaggregation was performed by additional sonication single pulse at 50% energy reduction than during Bioxome particle formation. For testing encapsulation efficacy, FACS measuring of Dextran-AF647 was performed after electroporated Bioxomes were incubated overnight with 5 μg latex beads (ThermoFisher Scientific).

### Adipose Tissue Derived Bioxomes:

Crude RNA encapsulated Adipose tissue derived Bioxomes were prepared—E8 Bioxomes were prepared from E6 cell culture of human adipose tissue and encapsulated by gentle ultrasonication with single six second pulse and 40% of energy and 0.5 mcg RNA (to preserve RNA integrity). The polydispersity index PDI of obtained RNA encapsulated Bioxomes was 548 nm. RNA encapsulated Bioxomes were further diluted ten times and then sonicated for another 30 sec pulse resulted in average particle size of 450 nm. Further extrusion by Avant extruder is optional to reach the size of 100 nm, especially if liver targeting is desirable.

### Example 14: Bioactivity of Bioxome/RNA Treatment on HFF—Human Foreskin Fibroblasts Culture

MTT-a 3-(4,5-dimethylthiazol-2-yl)-2,5-diphenyltetrazolium cell proliferation assay (Life Technologies) was carried out to investigate the effect of Adipose tissue derived Bioxomes loaded with 0.5 mcg internal RNA/per Bioxomes derived from E8 cells as described above on the cellular viability following starvation stress (serum deprivation). Briefly, 1×104 cells/well were seeded in 96-well plates and cultured for 18˜24 h to reach 90% confluency. Following attachment, cells were washed twice with PBS, then serum-free medium added. Both serum-deprived and control (10% serum) cells were harvested at 24 h. The cell culture supernatants were discarded and 20 μl MTT solution was added to each well (0.5 mg/ml; Sigma Aldrich; Merck KGaA, Darmstadt, Germany), then the cells were cultured for further 4 hours. The supernatants were then removed and 200 μl DMSO was added to each well, with slight agitation for 15 min. The absorbance at a wavelength of 490 nm was then detected with 4 replicates used for each well and a mean value calculated. Following FBS starvation from 10% DMEM FBS to 0% FBS was performed 24 hours prior to the experiment and samples treated with Bioxomes same proliferative effect on HFF similar to positive control (10% FBS) in comparison to serum free samples where viability was reduced to almost 30%.

### Example 15: In Vitro Scratch Assay on HFK

Bioxomes were prepared from primary Human Foreskin Keratinocytes—HFF cells by the described above process. The HFK were used as cells for in-vitro functional assay of wound healing model. On day 1 the cells were thawed from primary stock (P0-1) and cultured P1-2 on regular medium supplemented with 5-% FBS (preferably certified as exosomes depleted), and 1% Glutamax. On Day 2-3, the subculture was expanded to reach confluence for the experiment. Then cells were reseeded on 1.5% of FBS by split into 12-well inserts with diameter of 1.4 cm having diameter pores at a density of 2×106/cm² (Greiner), or 6-well plate (Corning) depending on the scratch assay kit protocol, or 8-chamber slides of each 0.75 cm×0.95 cm (Nunc). At Day 4 (˜1 day after subculture)—FBS was reduced down to 0.5% for 12 hours. Full FBS starvation was performed overnight in conjunction of the scratch performed by glass Pasteur pipette or by kit form template (Cytoselect wound healing assay, Cell Biolabs Incorporated, CBA-120). Cells were treated with FBS 5% in quadruplicates or triplicates and were left during all the adjustment procedure prior to the FBS starvation as a positive control and the same number of wells untreated by Bioxomes used as a negative control. Bioxomes were added after washing with HESS plus Hepes, post-collection of conditioned media that was used as a source of active Bioxomes/exosome released during starvation. Cell counting was performed as percentage of cell count vs positive control.

### Results:

FBS 5%-supplemented positive control reached full closure of the scratch at 24 hours. Untreated cells under full starvation and scratch stress stopped growing and underwent apoptosis at 24 hours. Viable cells were counted 12 hours after the scratch. Treatment with Bioxomes of HFF at 10sup5 reach 60-75% of the positive control. Bioxomes at the same dose prepared from HFF plus RNA from HFK of Passage 4 results in the same cell number. HFF Bioxomes with HFK RNA at all doses closed the scratch fully at 24 hours at the same rate as the positive control. The scratch trace remained seen in HFF Bioxomes without HFK RNA at 10sup3 HFF Bioxomes with HFF RNA performed similar to the HFF without RNA at high dose.

### Example 16: Pilot Study to Test Hepatoprotective Effect of Redoxomes in Liver Fibrosis In Vivo Model

To develop Bioxomes and Redoxomes as optional hepatoprotective treatments, Concovalin A (ConA)-induced liver necrosis was selected as a pathological in-vivo model. To test the efficacy of Bioxomes and Redoxomes in-vivo, n=36 of SD rats at the age of 10-11 weeks at study initiation were divided into 3 groups, 12 rats in each group. An additional group of six animals was used for pilot biodistribution study. Control group was treated by PBS, post ConA injection; and the hepatoprotective effect of Bioxomes and Redoxomes was calculated and presented as percentage of control. 20 mg/kg of ConA was injected intravenously to all animals to induce liver damage in comparison to basal nontreated animals. Single dose of E5 Bioxomes with fluorescent labelled ceramide BioDipy was injected IV following ConA administration. BioDipy ceramide was selected as lipid sensor due to the fact that lipid peroxidation at the site of inflammation is known to influence the release of the cargo at the target site. Rats from the biodistribution study were sacrificed eight hours after ConA and Bioxome BioDipy injection.

### Results:

Strong fluorescence was seen 2 hours post injection in liver, kidney and lungs. Liver was a major target organ for Bioxom BioDipy. To monitor functional parameters of acute liver damage, blood levels of alanine aminotransferase (ALT) were measured at 8 hours post ConA injection in all groups. ALT levels on ConA control group ranged from 300 to 1000 Units/Liter while basal level of ALT was lower than 100 Units/Liter. Liver necrosis was examined by hematoxylin and eosin (H&E) staining. For histological examination, a piece of the liver from each animal was trimmed and fixed by immersion in 10% buffered formalin for 24 hours, following graded ethanol dehydration. The blocks were further embedded in paraffin wax. Serial 3 mm sections were stained with H&E. The histopathological scoring valuated to simplify by two “blinded” pathologists by three grades: 0 basal/healthy; 1 low-to-moderate damage 3 toxic-to-tissue necrotic. Most Redoxome and Bioxome treated animals evaluated at score 2, proving hepatoprotective feasibility for further dose and time dependency in acute and chronic liver pathology models.

### Example 17: Pilot Study to Test Hepatoprotective Effect of Redoxomes in Liver Fibrosis In Vivo Model

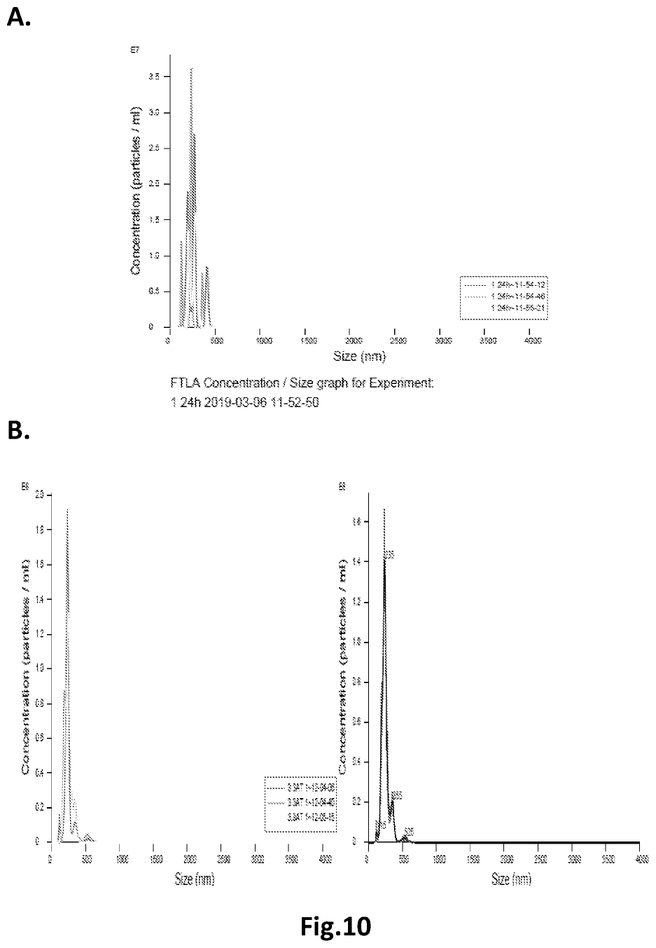

Various cell cultures and starting extraction cell raw material (form 2E3 to E9 cells) were used to prepare Bioxomes from stem, cell lines, and differentiated cells and cultured cells of plant cell origin. Yield of bioxome particles was found to correlate with starting cell number and varied from E6-E12 bioxome particles. Various industrial drying methods were used including, standard rotor evaporation, nitrogen and argon gas evaporation and freeze drying. Similar yield and particle size distribution were obtained by various methods. Cells were grown on adhesive cultures such as all used stem cells, HFF, HFK, HepG2, primaries, tobacco cells, and Jurkat (ATCC; Clone E6-1), that are CD3 expressing T-lymphocytes grown in bioreactors. Relevant cargo was added prior to sonication step or during extrusion as needed, at hydrophilic excipient buffer. To improve encapsulation efficiency, three freeze thaw cycles at least 24 hours apart were performed, resulting in the particle size <500 nm, when prepared from bone marrow stem cells. FIG. 10A demonstrates less uniform but still under QC specs, particle size data.

Solvents pharmaceutical grade, USP grade, >90% of purity or analytical grade were used. Bioxome particles were extracted from collected, liquid nitrogen banked, washed twice to remove FBD, or/and DMSO-containing cryopreservation medium, thawed pellets. Bioxome extraction was performed from fresh pellets and by direct extraction from various adhesive cultures cell layers (to avoid trypsin stress), from stem cells, stromal and epithelial cells and from primary, immortalized and cell lines. >E9 bioxome particles with representative uniform particle size were obtained from human adipose tissue isolated MSC, as demonstrated in FIG. 10B. HIP 3:2 was found to be optimal solvent system. It was used with 2:1 with RNAsave or RNAse free sterile water to co-precipitate RNA at single step. Pure RNA was recovered, concertation measured by Nanodrop to ensure purity and the integrity was tested. Typical concentration of RNA isolated by the process correlated with bioxome particle concentration is presented in the Table 1, standardized per cell number and cell weight:

| | | |
|---|---|---|
| | RNA total | Number of |
| Yield parameter | extraction | Particles |

| | | | |
|---|---|---|---|
| Cell Number | ~1-10 | mcg/E6 | E9/E6 |
| Cell wet weight | 1.2 | mcg/mg wwt | E8/mg wwt |

Bioxomes were prepared form conditioned medium by similar procedure with an additional washing step. Before collection of Bioxomes, cells were washed with HESS plus Hepes. At FBS depleted conditions high yield of RNA was collected.

Various molecules were used as a cargo: ascorbic acid, desferrioxamine and EDTA—as a models of small molecules hydrophilic cargo; RNA and green fluorescein protein—as a biological molecule; ceramides, tocopherol, docoshexaenoic acid ester, sphingomyelin and terpen—as bioactive lipid samples. Representative particle size of bioactive lipid encapsulated redoxome with bio-membrane modification and complex cargo resulted typically in particle size between average size 0.5-3 micron.

### Example 18: RNA Encapsulation and Electroporation Experiments

To prove that Bioxomes are feasible carriers for RNA transfection and delivery, Bioxomes were prepared from human red blood cells RBC. RBCs were collected from Group 0 blood samples, then RBCs separated from plasma and white blood cells by using centrifugation and leukodepletion filters (Terumo Japan). Bioxome extraction procedure was performed as described above. Electroporation calibration was done for future transfer of purified oligonuceotides into Bioxomes by validated positive control. The electroporation experiments were performed using a Gene Pulser Xcell electroporator (BioRad), exponential program at a fixed capacitance of 100 μF with 0.4 cm cuvettes. E12 Bioxomes obtained from E9 RBCs diluted in OptiMEM (ThermoFisher Scientific) and mixed with 4 μg Dextran conjugated with AF647 (ThermoFisher Scientific) to a total volume of 200 μl, 100 μl of Bioxomes aliquots were added to each cuvette and incubated on ice for 15 min at 150-250V. In a case that aggregated formed, deaggregation was performed by additional sonication single pulse at 50% energy reduction than during Bioxome particle formation as above. For testing encapsulation efficacy, FACS measuring of Dextran-AF647 was performed after electroporated Bioxomes were incubated overnight with 5 μg latex beads (ThermoFisher Scientific).

In addition, crude RNA encapsulated Adipose tissue derived Bioxomes were prepared—E8 Bioxomes were prepared from E6 cell culture of human adipose tissue and encapsulated by gentle ultrasonication with single six second pulse and 40% of energy and 0.5 mcg RNA (to preserve RNA integrity) and then encapsulated. The polydispersity index PDI of obtained RNA encapsulated Bioxomes was 548 nm. RNA encapsulated bioxome particles were further diluted ten times and then sonicated for another 30 sec pulse resulting in average particle size of 450 nm. Further extrusion by Avant extruder led to the average particle size of 100 nm, particularly for liver targeting. The robustness of process was validated by the efficient total RNA isolation at the same step, and similar yield of RNA was obtained from the conditioned medium.

### Example 19: Bioactivity of Bioxome/RNA Treatment on HFF—Human Foreskin Fibroblasts Culture

MTT-a 3-(4,5-dimethylthiazol-2-yl)-2,5-diphenyltetrazolium cell proliferation assay (Life Technologies) was carried out to investigate the effect of Adipose tissue derived Bioxomes loaded with 0.5 mcg internal RNA/per bioxomes derived from E⁸ cells as described above on the cellular viability following starvation stress (serum deprivation). 1×10⁴ cells/well were seeded in 96-well plates and cultured for 18˜24 h to reach 90% confluency. Following attachment, cells were washed twice with PBS, then serum-free medium added. Both serum-deprived and control (10% serum) cells were harvested at 24 h. The cell culture supernatants were discarded and 20 μl MTT solution was added to each well (0.5 mg/ml; Sigma Aldrich; Merck KGaA, Darmstadt, Germany), then the cells were cultured for a further 4 h. The supernatants were then removed and 200 DMSO was added to each well, with slight agitation for 15 min. The absorbance at a wavelength of 490 nm was then detected with 4 replicates used for each well and a mean value calculated. Following FBS starvation from 10% DMEM FBS to 0% FBS was performed 24 hours prior to the experiment and samples treated with Bioxomes same proliferative effect on HFF similar to positive control (10% FBS) in comparison to serum free samples where viability reduced to almost 30%.

### Example 20: Stability Proof of Concept Results

In order to test the feasibility for the stability design of Bioxomes, bone marrow and other various source cells-derived Bioxomes were prepared as described above and stored post-sonication at various temperatures, for 4° C. for one day, week, and month; stored at Revco −70° C. The concentration and size distribution of EVs were quantified using a NanoSight Tracking Analysis NS300 system (Malvern, UK). FIG. 3A-C represents examples of particle size. It was shown that particle concentration was not affected at short term (less than a week) storage, and aggregated after a month storage at 4° C. The stability of samples after freezing at 70° C. was not affected. Notably, that pre-sonicated Bioxomes were also stable after −70° C. at the same level as those who were repetitively sonicated prior to particle size measurement.

The terminology used herein is for the purposes of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

“Exosomes”, as the term is used herein, refers to membrane-derived microvesicles, which includes a range of extracellular vesicles, including exosomes, microparticles and shed microvesicle shed microvesicles, oncosomes, ectosomes, s secreted by many cell types under both normal physiological and pathological conditions and can be applied to intracellular vesicles, plant secretome vesicles, microbiome and retroviral-like particles of all sizes.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

Whenever the term “about,” is used, it is meant to refer to a measurable value such as an amount, a temporal duration, and the like, and is meant to encompass variations of +25%, +20%, +10%, +5%, +1%, or +0.1% from the specified value, as such variations are appropriate to perform the disclosed methods.

Throughout this application, various embodiments of this invention may be presented in a range format. It should be understood that, the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

By “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. An artificial bioxome particle comprising a cell membrane component and designed to undergo fusion with a target cell, wherein said bioxome particle is engineered to carry a cargo comprising at least one predetermined active molecule; and wherein said cargo can be released into the target cell after the fusion of the bioxome particle with the target cell; and wherein the cell membrane component is derived from a selected cellular or extracellular source.

2. The bioxome particle of claim 1, wherein the cargo comprises at least two active molecules.

3. The bioxome particle of claim 1 or 2, wherein the cargo comprises a plurality of active molecules.

4. The bioxome particle of any one of claims 1 to 3, wherein the source is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, biological fluids, excrement or surgery extracted tissues, milk, saliva, mucus, blood plasma, urine, feces, sebum, postnatal umbilical cord, placenta, amniotic sac, kidney tissue, neurological tissue, adrenal gland tissue, mucosal epithelium, smooth muscle tissue, a bacterial cell, a bacterial culture, a whole microorganism, conditional medium, amniotic fluid, lipoaspirate, liposuction byproducts, and a plant tissue.

5. The bioxome particle of any one of claims 1 to 4, wherein the active molecule is selected from the group consisting of nucleic acid, peptide, amino acid, polypeptide, nucleoside, growth factor, organic molecule, polyphenol, steroid, lipophilic poor soluble drug, inorganic molecule, anti-oxidant, hormone, antibody, vitamin, cytokine, enzyme, heat shock protein, or a combination thereof.

6. The bioxome particle of claim 5, wherein the active molecule is selected from cannabinoid, cannabinoid acid, and endocannabinoid.

7. The bioxome particle of claim 5 or 6; wherein the cannabinoid, cannabinoid acid, and endocannabionoid is selected from the group consisting of tetrahydrocannabinolic acid (THCa), cannabidiolic acid (CBDa), cannabinolic acid (CBNa) cannabichromenic acid (CBCa), tetrahydrocannabinol (THC), cannabinol (CBN), cannabidiol (CBD), and cannabichromene (CBC), acylethanolamides.

8. The bioxome particle of claim 5, wherein the nucleic acid is ribonucleic acid (RNA) or deoxyribonucleic acid (DNA).

9. The bioxome particle of claim 8, wherein the nucleic acid is RNA, and is selected from the group consisting of siRNA, an antisense RNA, iRNA, microRNA, an antagomir, an aptamer, and a ribozyme mRNA.

10. The bioxome particle of any one of claims 1 to 9, wherein the active molecule has a therapeutic effect.

11. The bioxome particle of claim 10, wherein the therapeutic effect is selected from the group consisting of anti-inflammatory effect, anti-fibrotic effect, anti-tumor effect, and neuroprotective effect.

12. The bioxome of any one of claims 1 to 11 which is a redoxome, wherein the cargo comprises at least one redox active free-radicals scavenging compound.

13. The redoxome of claim 12, comprising fenton reaction complex blockers, hydroxyl radical trap, iron chelator and a lipid radical trap.

14. The redoxome of claim 12 or 11, capable of blocking LPO chain reaction, such as lipid radical/peroxide trap, such as vitamin E, terpenoids, polyphenols, flavonoid, phenolic acids, cannabinoids, retinoids, vitamin D, lipoic acid, sterols.

15. The redoxome of claim 13 or 14, wherein the radical trap is ascorbic acid, nitric oxid donor (S-nitrosoglutathione), or a derivative thereof.

16. The redoxome of any one of claims 12 to 15, wherein the iron chelator is selected from the group consisting of desferrioxamine (DFX), ethylenediaminetetraacetic acid (EDTA), rutin, disodium EDTA, tetrasodium EDTA, calcium disodium EDTA, diethylenetriaminepentaacetic acid (DTPA) or a salt thereof, hydroxyethlethylenediaminetriacetic acid (HEDTA) or a salt thereof, nitrilotriacetic acid (NTA), acetyl trihexyl citrate, aminotrimethylene phosphonic acid, beta-alanine diacetic acid, bismuth citrate, citric acid, cyclohexanediamine tetraacetic acid, diammonium citrate, dibutyl oxalate, diethyl oxalate, diisobutyl oxalate, diisopropyl oxalate, dilithium oxalate, dimethyl oxalate, dipotassium EDTA, dipotassium oxalate, dipropyl oxalate, disodium EDTA-copper, disodium pyrophosphate, etidronic acid, HEDTA, methyl cyclodextrin, oxalic acid, pentapotassium, triphosphate, pentasodium aminotrimethylene phosphonate, pentasodium pentetate, pentasodium triphosphate, pentetic acid, dicarboxyic acid, phytic acid, potassium citrate, sodium citrate, sodium dihydroxyethylglycinate, sodium gluceptate, sodium gluconate, sodium hexametaphosphate, sodium metaphosphate, sodium metasilicate, sodium oxalate, sodium trimetaphosphate, tea-EDTA, tetrahydroxypropyl ethylenediamine, tetrapotassium etidronate, tetrapotassium pyrophosphate, tetrasodium etidronate, tetrasodium pyrophosphate, tripotassium EDTA, trisodium EDTA, trisodium hedta, trisodium NTA, trisodium phosphate, malic acid, fumaric acid, maltol, succimer, penicillamine, dimercaprol, deferipron, a natural protein based iron chelator, melatonin, siderphore, zinc or copper cation, or salt or complex, and desferrioxamine mesylate, or a combination thereof.

17. The redoxome of claim 16, wherein the iron chelator is selected from the group consisting of EDTA (ethyl enediaminetetraacetic acid), DTPA (diethylene triamine pentaacetic acid), NTA (nitrilotriacetic acid), detoxamin, deferoxamine, deferiprone, deferasirox, glutathione, metalloprotein, ferrochel (bis-glycinate chelate), ceruloplasmin, penicillamine, cuprizone, trientine, ferrulic acid, zinc acetate, lipocalin 2, and dimercaprol.

18. The bioxome particle of any one of claims 1 to 17 which is a long circulating, slow release bioxome.

19. The bioxome of any one of claims 1 to 18 which is a selective targeting bioxome.

20. The bioxome of any one of claims 1 to 19 which is an immunogenic bioxome.

21. A composition comprising the bioxome particle of any one of claims 1 to 20 and at least one carrier.

22. The composition of claim 21, wherein the composition is a pharmaceutical composition and the carrier is a pharmaceutically acceptable carrier.

23. The composition of claim 21 or 22, wherein the composition is suitable for oral, intravenous, subcutaneous, intraperitoneal, sublingual, intra-tissue, through tissue inserted implant administration, intrathecal, intra-muscular, topical, ocular, intra-nasal, rectal, vaginal, pulmonary, transmucosal and transdermal administration.

24. The composition of claim 21, wherein the composition is a cosmeceutical composition and the carrier is a cosmeceutically acceptable carrier.

25. The composition of claim 21, wherein the composition is an edible composition, and the carrier is food grade carrier.

26. A process for the manufacture of a sample comprising a plurality of bioxome particles, wherein the bioxome particles are engineered to carry a cargo comprising at least one active molecule and designed to undergo fusion with a target cell to release the cargo; and wherein said bioxome particles comprise a cell membrane component derived from a selected cellular or extracellular source; the process comprising:
a. Performing total cell lipid extraction from the selected cellular or extracellular source in a mild solvent system to obtain a lipid extract;
b. Drying the lipid extract; and
c. Inducing self-assembly of bioxome particles by performing at least one step of ultra-sonication;
wherein the resulting bioxome particles in the sample are characterized by an average particle size of about 0.03 μm to 5 μm.

27. The process of claim 26, wherein the average particle size is 0.05 μm to 3 μm.

28. The process of claim 27, wherein the average particle size is 0.08 μm to 1.5 μm.

29. The process of any one of claims 26 to 28, wherein the sample comprising the bioxome particle has the pH of 3.5 to 5.5.

30. The process of claim 29, wherein the sample comprising the bioxome particle has the pH of 4.5 to 5.

31. The process of any one of claims 26 to 30, wherein the mild solvent system comprises a mixture of polar and non-polar solvents.

32. The process of any one of claim 31, wherein the polar solvent in the solvent system is selected from the group consisting of isopropanol, ethanol, n-butanol, and water-saturated n-butanol.

33. The process of any one of claim 31 or 32, wherein the non-polar solvent in the solvent system is selected from hexane, solvents from the terpene group, and supercritical CO² extraction.

34. The process of claim 33, wherein the non-polar solvent in the solvent system is n-hexane.

35. The process of claim 33, wherein the solvent from the terpene group is selected from the group consisting of d-limonene, α-pinene and para-cymene.

36. The process of any one of claims 31 to 34, wherein the polar solvent in the solvent system is isopropanol, and the non-polar solvent is n-hexane.

37. The process of any one of claims 26 to 36, wherein the solvent system further comprises a stabilizer.

38. The process of claim 37, wherein the stabilizer is butyl-hydroxytoluene (BHT) or a lipid radical trap.

39. The process of any one of claims 26 to 38, wherein the solvent system further comprises an antioxidant, a surfactant, vitamin E, squalene, cholesterol, or a combination thereof.

40. The process of any one of claims 26 to 39, further comprising the step of co-precipitation of a nucleic acid.

41. The process of claim 40, wherein the nucleic acid is ribonucleic acid (RNA) or deoxyribonucleic acid (DNA).

42. The process of claim 41, wherein the nucleic acid is RNA.

43. The process of any one of claims 26 to 42, wherein the cellular or extracellular source for total lipid extraction is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, postnatal umbilical cord, placenta, amniotic sac, kidney tissue, neurological tissue, adrenal gland tissue, mucosal epithelium, smooth muscle tissue, a bacterial cell, a bacterial culture, a whole microorganism, conditional medium, amniotic fluid, lipoaspirate, liposuction byproducts, and a plant tissue.

44. The process of any one of claims 26 to 43, wherein the lipid extraction is performed from cell-conditioned media, lyophilized conditioned cell media, cell pellet, frozen cells, dry cells, washed cell bulk, non-adhesive cell suspension, and adhesive cell layer.

45. The process of claim 44, wherein the adhesive cell layer is grown in cell culture plasticware selected from a (multi)flask, a dish, a scaffold, beads, and a bioreactor.

46. A sample comprising a plurality of bioxome particles, prepared according to the process of any one of claims 26 to 45.

47. A method of treating or preventing a pathology in a subject in need of such treatment, comprising administering to the subject the pharmaceutical composition of any one of claims 21 to 23.

48. The method of claim 47, wherein the pathology is selected from the group consisting of an inflammatory disorder, a neurological disorder, an infectious disorder, a malignancy, a disorder of the immune system, and an autoimmune disorder.

49. A method of improving a skin condition in a subject in need comprising administering to the subject the composition of any one of claims 21 to 24.

50. The method of claim 49, wherein the composition is administered topically.

51. Use of the bioxome particle of any one of claims 19 to 31 as a vehicle for delivery of active molecules to the target site.

52. The bioxome particle of any one of claims 1 to 20 for use as a medicament.

53. The bioxome particle of any one of claims 19 to 31 for use in the treatment of an inflammatory disorder, a neurological disorder, an infectious disorder, a malignancy, a disorder of the immune system, and an autoimmune disorder.
